(19) United States
(12) Patent Application Publication
(10) Pub. No.: US 2005/0032063 A1
(43) Pub. Date: Feb. 10, 2005

(54) Detection of matrix metalloproteinase rna in plasma and serum

(76) Inventor: Michael Kopreski, Long Valley, NJ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/497,634

(22) PCT Filed: Dec. 3, 2002
(86) PCT No.: PCT/US02/38719

Related U.S. Application Data
(60) Provisional application No. 60/337,550, filed on Dec. 3, 2001.

(51) Int. Cl. C12Q 1/68; C12P 19/34

(52) U.S. Cl. 435/6; 435/91.2

(57) ABSTRACT

The methods of the invention detect in a quantitative fashion matrix metalloproteinase RNA in blood plasma, serum, and other bodily fluids. The inventive methods are useful for aiding detection, diagnosis, monitoring, treatment, or evaluation of neoplastic disease.

### BACKGROUND OF THE INVENTION

This invention relates to methods for detecting and monitoring matrix metalloproteinase (MMP) RNA in bodily fluids such as blood plasma and serum. Matrix metalloproteinases are collagenases involved in the degradation and remodeling of the extracellular matrix that have an important role in the invasiveness and metastatic properties of a cancer. While MMP is overexpressed in many invasive and metastatic cancers, its expression is also upregulated in many premalignant tissues such as adenoma. MMP is thus expressed in many malignant and premalignant tissues, where in particular matrix metalloproteinase-1 (MMP-1; GenBank Accession No. NM_{—}001421), matrix metalloproteinase-2 (MMP-2; GenBank Accession No. BC_{—}002576), matrix metalloproteinase-3 (MMP-3; GenBank Accession No. NM_{—}002422), matrix metalloproteinase-7 (MMP-7; GenBank Accession No. NM_{—}002423), matrix metalloproteinase-9 (MMP-9; GenBank Accession No. NM_{—}004994), matrix metalloproteinase-10 (MMP-10; GenBank Accession No. NM_{—}002425), matrix metalloproteinase-11 (MMP-11; GenBank Accession No. NM_{—}005940), matrix metalloproteinase-12 (MMP-12; GenBank Accession No. NM_{—}002426), and matrix metalloproteinase-14 (MMP-14; GenBank Accession No. NM_{—}004955) are over-expressed in malignancy and/or premalignancy. In particular, MMP-7 is overexpressed in premalignant tissue such as adenoma (Heslin et al., Ann. Surg., 233: 786-792, 2001). Since ribonucleic acid (RNA) is essential for producing MMP protein, detection and monitoring of matrix metalloproteinase RNA (MMP RNA) provides a method for assessing and monitoring MMP gene expression, thereby providing a method for monitoring and determining the presence and propensity for MMP-expressing cells and tissue. MMP-expressing cells are particularly common to cancers having with a poor prognosis or metastatic tendency. MMP RNA being associated with cancer and premalignancy, is therefore characterized as a tumor-associated RNA herein, the detection of which provides a method for diagnosing, detecting, monitoring, prognosticating, and evaluating cancer and premalignancy.

Co-owned U.S. Pat. No. 6,329,179, issued Dec. 11, 2001, incorporated herein by reference in its entirety, provides a method for detecting tumor-associated RNA in bodily fluids such as blood plasma and serum, wherein said RNA detection is used for detecting, monitoring, or evaluating cancer or premalignant conditions.

MMP RNA being recognized herein as being tumor-associated RNA, there is a newly-appreciated need in the art to identify premalignant or malignant states in an animal, most preferably a human, by detecting in a qualitative or quantitative fashion MMP RNA such as MMP-1 RNA, MMP-2 RNA, MMP-3 RNA, MMP-7 RNA, MMP-9 RNA, MMP-10 RNA, MMP-1 1 RNA, MMP-12 RNA, and MMP-14 RNA, in bodily fluids such as blood plasma or serum. Further, there is a need in the art to evaluate the predisposition in an animal, most preferably a human, for diseases characterized by MMP RNA expression, by detecting MMP RNA in bodily fluids such as blood plasma or serum, wherein cancer is one such disease.

### SUMMARY OF THE INVENTION

The present invention describes a method of evaluating an animal, most preferably a human, for premalignant or malignant states, disorders or conditions by detecting MMP mRNA in bodily fluids, preferably blood and most preferably blood plasma and serum as well as in other bodily fluids, preferably urine, effusions, ascites, saliva, cerebrospinal fluid, cervical, vaginal, and endometrial secretions, gastrointestinal secretions, bronchial secretions, and associated tissue washings and lavages. Specific MMP RNA species are recognized to include MMP-1 (GenBank Accession No. NM_{—}001421), MMP-2 (GenBank Accession No. BC_{—}002576), MMP-3 (GenBank Accession No. NM_{—}002422), MMP-7 (GenBank Accession No. NM_{—}002423), MMP-9 (GenBank Accession No. NM_{—}004994), MMP-10 (GenBank Accession No. NM_{—}002425), MMP-11 (GenBank Accession No. NM_{—}005940), MMP-12 (GenBank Accession No. NM_{—}002426), MMP-14 (GenBank Accession No. NM_{—}004955) The invention provides the method of amplifying and detecting extracellular MMP RNA. In a preferred embodiment, the present invention provides a method for detecting MMP RNA in blood or a blood fraction, including plasma and serum, or in other bodily fluids, the method comprising the steps of extracting RNA from blood, plasma, serum, or other bodily fluid, in vitro amplifying in a qualitative or quantitative fashion one or more MMP mRNA or their cDNA, and detecting the amplified product of the MMP mRNA or its cDNA. Said amplification methods may further include the qualitative or quantitative comparison to a reference RNA species normally present in the plasma, serum, or bodily fluid of individuals with or without cancer.

In a first aspect of this embodiment, the present invention provides methods for detecting MMP RNA in blood or blood fractions, including plasma and serum, in a human or animal. Said methods are useful for detecting, diagnosing, monitoring, treating and evaluating various proliferative disorders, particularly stages of neoplastic disease, including premalignancy, early cancer, non-invasive cancer, carcinoma in-situ, invasive cancer and advanced cancer. Said methods are further advantageous for determining or predicting whether a cancer is or will become invasive, metastatic, or associated with poor prognosis. In this aspect, the method comprises the steps of extracting RNA from blood or blood plasma or serum, in vitro amplifying said MMP RNA comprising the extracted RNA either qualitatively or quantitatively, and detecting the amplified product of MMP RNA or its cDNA.

The invention in a second aspect provides a method for detecting MMP RNA in any bodily fluid. Preferably, said bodily fluid is whole blood, blood plasma, serum, urine, effusions, ascitic fluid, saliva, cerebrospinal fluid, cervical secretions, vaginal secretions, endometrial secretions, gastrointestinal secretions, bronchial secretions including sputum, secretions or washings from the breast, or other associated tissue washings or lavages from a human or animal. In this aspect, the method comprises the steps of extracting RNA from the bodily fluid, in vitro amplifying MMP RNA comprising a fraction of the extracted RNA, or preferably the corresponding cDNA into which the RNA is converted, in a qualitative or quantitative fashion, and detecting the amplified product of MMP RNA or cDNA. In these embodiments, the inventive methods are particularly advantageous for detecting, diagnosing, monitoring, treating or evaluating various proliferative disorders, particularly stages of neoplastic disease, including premalignancy, early cancer, non-invasive cancer, carcinoma-in-situ, invasive cancer and advanced cancer. Said methods are further advantageous for determining or predicting whether a cancer is or will become invasive, metastatic, or associated with poor prognosis.

The method of the invention is additionally useful for identifying MMP RNA-expressing cells or tissue in an animal, most preferably a human. In these embodiments, detection of an in vitro amplified product of MMP RNA using the inventive methods indicates the existence of MMP RNA-expressing cells or tissue in an animal, most preferably a human.

The invention provides primers and probes useful in the efficient amplification of extracellular MMP mRNA or cDNA from bodily fluid, most preferably blood plasma or serum.

The invention further provides a diagnostic kit for detecting MMP RNA in bodily fluid, preferably blood plasma or serum, wherein the kit comprises primers, probes or both primers and probes for amplifying and detecting extracellular MMP RNA or cDNA derived therefrom, and instructions on the use thereof with the methods of the invention.

In preferred embodiments of the inventive methods, MMP RNA is extracted from whole blood, blood plasma or serum, or other bodily fluids using an extraction method such as but not limited to gelatin extraction method; silica, glass bead, or diatom extraction method; guanidinium thiocyanate acid-phenol based extraction methods; guanidinium thiocyanate acid based extraction methods; methods using centrifugation through cesium chloride or similar gradients; phenol-chloroform based extraction methods; or other commercially available RNA extraction methods. Extraction may further be performed using probes that specifically hybridize to MMP RNA.

In preferred embodiments of the inventive methods, MMP RNA or cDNA derived therefrom is amplified using an amplification method such as reverse transcriptase polymerase chain reaction (RT-PCR); ligase chain reaction; signal amplification such as DNA signal amplification; amplifiable RNA reporters; Q-beta replication; transcription-based amplification; isothermal nucleic acid sequence based amplification; self-sustained sequence replication assays; boomerang DNA amplification; strand displacement activation; cycling probe technology; cleavase-based amplification; or any combination or variation thereof.

In preferred embodiments of the inventive methods, detecting an amplification product of MMP RNA or MMP cDNA is accomplished using a detection method such as gel electrophoresis; capillary electrophoresis; conventional enzyme-linked immunosorbent assay (ELISA) or modifications thereof, such as amplification using biotinylated or otherwise modified primers; nucleic acid hybridization using specific, detectably-labeled probes, such as fluorescent-, radioisotope-, or chromogenically-labeled probe; Northern blot analysis; Southern blot analysis; electrochemiluminescence; reverse dot blot detection; and high-performance liquid chromatography.

In particularly preferred embodiments of the inventive methods, MMP RNA is converted to cDNA using reverse transcriptase following extraction of RNA from a bodily fluid and prior to amplification.

The methods of the invention are advantageously used for providing a diagnosis or prognosis of, or as a predictive indicator for determining a risk for an animal, most preferably a human, for developing a proliferative, premalignant, neoplastic or malignant disease comprising or characterized by the existence of cells over expressing MMP RNA. The methods of the invention are particularly useful for providing a diagnosis for identifying humans at risk for developing or who have developed malignancy or premalignancy and for determining predisposition to malignancy. Most preferably, the malignant or premalignant diseases, conditions or disorders advantageously detected, diagnosed, or inferred using the methods of the invention are breast, prostate, ovarian, lung, cervical, colorectal, gastric, hepatocellular, pancreatic, bladder, endometrial, kidney, skin, and esophageal cancers, and premalignancies and carcinoma in-situ such as prostatic intraepithelial neoplasia (PIN), cervical dysplasia, cervical intraepithelial neoplasia (CIN), bronchial dysplasia, atypical hyperplasia of the breast, ductal carcinoma in-situ, colorectal adenoma, atypical endometrial hyperplasia, and Barrett's esophagus.

In certain preferred embodiments of the methods of the invention, MMP RNA or cDNA derived therefrom is amplified in a quantitative manner, thereby enabling the quantitative comparison of MMP RNA present in a bodily fluid such as blood plasma or serum from an animal, most preferably a human. In these embodiments, the amount of extracellular MMP RNA detected in an individual are compared with a range of amounts of extracellular MMP RNA detected in said bodily fluid in populations of animals known to have a premalignant, neoplastic, or malignant disease, most preferably a particular premalignant, neoplastic, or malignant disease. Additionally, the amount of extracellular MMP RNA detected in an individual is compared with a range of amounts of extracellular MMP RNA detected in said bodily fluid in populations of animals known to be free from a premalignant, neoplastic, or malignant disease. In particularly preferred aspects of this embodiment, comparison of MMP RNA is further made to a reference RNA extracted, amplified, and detected from said bodily fluid, wherein said reference RNA is not MMP RNA, but preferably an RNA normally present in the bodily fluid of both healthy individuals and those with cancer. In another aspect, said reference RNA is not MMP RNA, but is an RNA present in the bodily fluid of individuals with cancer.

The methods of the invention further provide ways to identify individuals having a MMP over expressing malignancy or premalignancy, thereby permitting rational, informed treatment options to be used for making therapeutic decisions. In particular, the methods of the invention are useful in identifying individuals having a premalignancy or malignancy that might benefit from a MMP-directed therapy such as but not limited to a matrix metalloproteinase inhibitor, either alone or administered with therapeutically-effective amounts of other chemotherapeutic or anticancer drugs. This aspect of the invention is further useful in identifying individuals for chemopreventive therapies, whether said therapy is directed at the MMP or not.

Another advantageous use for the methods of the invention is to provide a marker for assessing the adequacy of anticancer therapy, including surgical intervention, chemotherapy, biotherapy including vaccine therapy and therapy with monoclonal agents, antisense therapy, or radiation therapy, administered preventively or palliatively, or for determining whether additional or more advanced therapy is required. The invention therefore provides methods for developing a prognosis in such patients both prior to therapy and following therapy.

The methods of the invention also allows identification or analysis of MMP RNA, either qualitatively or quantitatively, in the blood or other bodily fluid of an individual, most preferably a human who has completed therapy, as an early indicator of relapsed cancer, impending relapse, or treatment failure.

Specific preferred embodiments of the present invention will become evident from the following more detailed description of certain preferred embodiments and the claims.

### DETAILED DESCRIPTION OF THE INVENTION

The invention provides methods for detecting MMP RNA, such as MMP-1 RNA, MMP-2 RNA, MMP-3 RNA, MMP-7 RNA, MMP-9 RNA, MMP-10 RNA, MMP-11 RNA, MMP-12 RNA, and MMP-14 RNA in bodily fluids of an animal, most preferably a human, thereby enabling detection of cancerous or precancerous cells that overexpress MMP RNA in the human or animal.

In preferred embodiments of the methods of the invention, extracellular RNA containing MMP RNA is extracted from a bodily fluid. This extracted RNA is then amplified, either after conversion into cDNA or directly, using in vitro amplification methods in either a qualitative or quantitative manner using primers or probes specific for the MMP RNA or cDNA of interest. The amplified product or signal is then detected in either a qualitative or quantitative manner.

In the practice of the methods of the invention, MMP RNA may be extracted from any bodily fluid, including but not limited to whole blood, plasma, serum, urine, effusions, ascitic fluid, saliva, cerebrospinal fluid, cervical secretions, vaginal secretions, endometrial secretions, gastrointestinal secretions, bronchial secretions including sputum, breast fluid, or secretions or washings or lavages, using, for example, extraction methods described in co-owned U.S. Pat. No. 6,329,179, issued Dec. 11, 2001, the entire disclosure of which is hereby incorporated by reference. In a preferred embodiment, the bodily fluid is either blood plasma or serum. It is preferred, but not required, that blood be processed soon after drawing, and preferably within three hours, as to minimize any nucleic acid degradation in the sample. In a preferred embodiment, blood is first collected by venipuncture and may be kept on ice until use. Preferably, within 30 minutes to one hour of drawing the blood, serum is separated by centrifugation, for example at 1100×g for 10 minutes at 4° C. When using plasma, the blood is not permitted to coagulate prior to separation of the cellular and acellular components. Serum or plasma can be frozen, most preferably at −70° C. after separation from the cellular portion of blood until further assayed. When using frozen blood plasma or serum, the frozen serum or plasma is rapidly thawed, for example in a 37° C. water bath, and RNA is extracted therefrom without delay, most preferably using a commercially-available kit (for example, Perfect RNA Total RNA Isolation Kit, obtained from Five Prime-Three Prime, Inc., Boulder, Colo.) according to manufacturer's instructions but as applied to bodily fluid. Other methods of RNA extraction are further provided in co-owned U.S. Pat. No. 6,329,179, issued Dec. 11, 2001, incorporated herein by reference in its entirety.

Following the extraction of RNA from a bodily fluid, a fraction of which contains MMP mRNA, the MMP mRNA or cDNA derived therefrom is amplified in vitro. Applicable amplification assays are detailed in co-owned U.S. Pat. No. 6,329,179, issued Dec. 11, 2001, as herein incorporated by reference, and include but are not limited to reverse transcriptase polymerase chain reaction (RT-PCR), ligase chain reaction, nucleic acid signal amplification methods including but not limited to branched chain signal amplification, amplifiable RNA reporters, Q-beta replication, transcription-based amplification, boomerang DNA amplification, strand displacement activation, cycling probe technology, cleavase-based amplification, isothermal nucleic acid sequence based amplification, and other self-sustained sequence replication assays.

In preferred embodiments of the methods of the invention, MMP mRNA is converted into cDNA using reverse transcriptase prior to in vitro amplification using methods known in the art. For example, a sample, such as 10 microL extracted serum RNA is reverse-transcribed in a 30 microL volume containing 200 Units of Moloney murine leukemia virus (MMLV) reverse transcriptase (Promega, Madison, Wis.), a reaction buffer supplied by the manufacturer, 1 mM dNTPs, 0.5 micrograms random hexamers, and 25 Units of RNAsin (Promega, Madison, Wis.). Reverse transcription is typically performed under an overlaid mineral oil layer to inhibit evaporation and incubated at room temperature for 10 minutes followed by incubation at 37° C. for one hour.

Alternatively, other methods well known in the art can be used to reverse transcribe MMP RNA to cDNA, including but not limited to those provided in these references incorporated herein by reference in their entirety, or by oligodT or primer-specific methods of reverse transcription.

Amplification primers are specific for amplifying MMP RNA or cDNA. In a preferred embodiment, amplification of MMP-1 cDNA is performed by RT-PCR, preferably as set forth in Shiozawa et al. (Mod. Pathol. 13:925-33, 2000), incorporated herein by reference in its entirety. In these embodiments, the preferred oligonucleotide primer sequences are as follows:

(MMP-1 sense; SEQ ID No. 1)

| | |
|---|---|
| Primer 1: | 5′-TTCATTTCTGTTTTGTGGCC-3′ |

(MMP-1 antisense; SEQ ID No. 2)

| | |
|---|---|
| Primer 2: | 5′-ATTTTTCCTGCAGTTGAACC-3′. |

In one example of a preferred embodiment of the invention, MMP-1 RNA is harvested from approximately 1.75 mL aliquots of serum or plasma, and RNA extracted therefrom using the Perfect RNA Total RNA Isolation Kit (Five Prime-Three Prime) or similar commercial extraction kit. From this extracted RNA preparation, 10 microL are then reverse transcribed to cDNA as described above. RT-PCR for the MMP-1 cDNA is performed using 5 microL of MMP-1 cDNA in a final volume of 50 microL in a reaction mixture containing 1U of Amplitaq Gold Perkin Elmer Corp., Foster City, Calif.), a reaction buffer provided by the Amplitaq supplier, 1.5 mM MgCl₂, 200 microM each dNTP, and 25 picomoles each of appropriate primer as identified above (Primer 1 and 2 for MMP-1, SEQ ID No. 1 and SEQ ID No. 2). The mixture is then amplified in a single-stage reaction in a thermocycler under a temperature profile consisting of an initial 2 minute incubation at 94° C., followed by 45 cycles of denaturation at 94° C. for 60 seconds, annealing at 52° C. for 90 seconds, and extension at 72° C. for 60 seconds, followed by a final extension at 72° C. for 5 minutes. Detection of the amplified product is then achieved, for example, by gel electrophoresis through a 1.5% agarose gel, using ethidium bromide staining for visualization and identification of the product fragment. Alternatively, the amplified products may thereafter be hybridized to end-labeled oligonucleotide probes and detected, or detected by other methods as previously described.

The invention provides for alternative methods of amplification of MMP RNA or cDNA known in the art, including but not limited to the methods of Hagemann et al. (Eur. J. Cancer 37: 1839-46, 2001) for amplification of MMP-1 cDNA, MMP-2 cDNA, MMP-3 cDNA, MMP-9 cDNA, MMP-11 cDNA, MMP-12 cDNA, and MMP-14 cDNA; the method of Yamashita et al. (Br. J. Cancer 84: 276-82, 2001) for amplification of MMP-1 cDNA; and the methods of Heslin et al. (Ann. Surg. 233: 786-792, 2001) for amplification of MMP-2 cDNA, MMP-7 cDNA, and MMP-9 cDNA; these references incorporated herein by reference in their entirety. Alternative methods of amplification further include but is not limited to signal amplification methods, ligase chain reaction, amplifiable RNA reporters, Q-beta replication, transcription-based amplification, isothermal nucleic acid sequence based amplification, self-sustained sequence replication assays, boomerang DNA amplification, strand displacement activation, cycling probe technology, cleavase-based amplification, and combinations or variations thereof. Amplification methods can further be performed in qualitative or quantitative fashion using primers specific for an internal control sequence of a reference RNA, such as glyceraldehyde-3-phosphate dehydrogenase or beta-actin, as described in said references, wherein said controls are RNA present in the bodily fluid of both healthy individuals and individuals with cancer.

In a particularly preferred embodiment, MMP RNA or cDNA is amplified in a quantitative amplification reaction. Quantitative amplification of MMP RNA or cDNA is particularly advantageous because this method enables statistically-based discrimination between patients with neoplastic disease and populations without neoplasm, including normal individuals. Using these methods, quantitative distributions of MMP RNA in bodily fluids such as blood plasma or serum are established in populations with neoplastic diseases, and in normal populations. Using this population information, the amount of extracellular MMP RNA in an individual is compared with the range of amounts of extracellular MMP RNA in said populations, resulting in a determination of whether the detected amount of extracellular MMP RNA in an individual indicates that the individual has a premalignant, neoplastic or malignant disease, or has a predisposition to developing such a disease.

In alternative preferred embodiments, amplified products can be detected using other methods, including but not limited to gel electrophoresis; capillary electrophoresis; ELISA or modifications thereof, such as amplification using biotinylated or otherwise modified primers; nucleic acid hybridization using specific, detectably-labeled probes, such as fluorescent-, radioisotope-, or chromogenically-labeled probe; Southern blot analysis; Northern blot analysis; electrochemiluminescence; reverse dot blot detection; and high-performance liquid chromatography. Furthermore, detection may be performed in either a qualitative or quantitative fashion.

PCR product fragments produced using the methods of the invention can be further cloned into recombinant DNA replication vectors using standard techniques. RNA can be produced from cloned PCR products, and in some instances the RNA expressed thereby, using the TnT Quick Coupled Transcription/Translation kit (Promega, Madison, Wis.) as directed by the manufacturer.

The methods of the invention as described above can be performed in like manner for detecting MMP mRNA from other bodily fluids, including but not limited to whole blood, urine, effusions, ascitic fluid, saliva, cerebrospinal fluid, cervical secretions, vaginal secretions, endometrial secretions, gastrointestinal secretions, breast fluid or secretions, and bronchial secretions including sputum, and from washings or lavages. Whereas fractionation of the bodily fluid into its cellular and non-cellular components is not required for the practice of the invention, the non-cellular fraction may be separated, for example, by centrifugation or filtration of the bodily fluid.

The methods of the invention as described above can be performed in like manner as would be understood in the art to demonstrate single nucleotide polymorphism in the MMP gene by detecting said polymorphism in plasma, serum, or other bodily fluid, one such preferred method being the method of Zhu et al. (Cancer Res., 61: 7825-9, 2001), incorporated herein by reference in its entirety.

The methods of the invention are thereby useful in the practice of a diagnostic method for detecting MMP mRNA in an animal, most preferably a human at risk for developing or who has developed a premalignant, neoplastic or malignant disease consisting of cells over expressing MMP mRNA. The invention further provides a method of identifying humans at risk for developing, or who have developed premalignancies or cancer, including but not limited to cancers of the breast, prostate, ovary, lung, cervix, colon, rectum, stomach, liver, pancreas, bladder, endometrium, kidney, brain, skin including squamous cell cancer and malignant melanoma, and esophagus, as well as premalignancies and carcinoma in-situ including but not limited to prostatic intraepithelial neoplasia (PIN), cervical dysplasia and cervical intraepithelial neoplasia (CIN), bronchial dysplasia, atypical hyperplasia of the breast, ductal carcinoma in-situ, colorectal adenoma, atypical endometrial hyperplasia, and Barrett's esophagus.

The diagnostic methods and advantageous applications of the invention can be performed using a diagnostic kit as provided by the invention, wherein the kit includes primers specific for MMP cDNA synthesis or in vitro amplification or both, and/or specific probes for detecting MMP RNA, cDNA or in vitro amplified DNA fragments or amplified signals thereof. The kit may further include methods and reagents for extracting MMP RNA from an extracellular bodily fluid, wherein the bodily fluid includes but is not limited to plasma or serum.

The inventive methods have significant utility in assigning and monitoring non-specific therapies, including anti-neoplastic therapies such as chemotherapy, radiation, and surgery, or specific therapies such as antisense therapies, vaccines, monoclonal antibody therapy, and MMP-directed therapeutic agents such as matrix metalloproteinase inhibitors. The inventive methods are also useful for monitoring response, relapse, and prognosis of MMP producing neoplastic diseases. Of particular value, the invention allows a determination that a therapy is therapeutically indicated even in cases of premalignancy, early cancer, occult cancer or minimum residual disease are present. Thus, the invention permits selection of patients for said therapies or monitoring of therapeutic intervention, including chemoprevention, when tumor burden is low or when malignancy has not yet developed.

The invention further enables MMP RNA to be evaluated in blood plasma or serum or other bodily fluid in combination with detection of other tumor-associated or tumor-derived RNA or DNA, including hypermethylated or aberrantly methylated DNA, microsatellite DNA, mutated or altered oncogenes or tumor suppressor genes, and/or other overexpressed RNA, in a concurrent or sequential fashion, such as in a multiplexed assay or in a chip-based assay, thereby increasing the sensitivity or efficacy of the assay in the detection or monitoring of neoplastic diseases, or in monitoring and evaluating MMP-dependent processes or prognostic indicators.

The methods of the invention and preferred uses for the methods of the invention are more fully illustrated in the following Example. This Example illustrates certain aspects of the above-described method and advantageous results. This Example is shown by way of illustration and not by way of limitation.

### EXAMPLE 1

A 52 year old man with a family history of colorectal cancer undergoes a cancer screening test by providing a blood plasma sample for a multiplexed assay that includes evaluation of the plasma for MMP RNA. MMP RNA is evaluated by the methods of the invention in a quantitative manner as described. In addition, other tumor-associated nucleic acids, including K-ras DNA and hTERT RNA, will be evaluated by the multiplexed assay. The assay indicates MMP RNA is present in the plasma at levels higher than expected in the normal population. In addition, the multiplexed assay is positive for mutated K-ras oncogene present in the plasma, and for hTERT RNA. Overall, the assay results would be consistent with neoplasia. The man would subsequently undergo a conventional colonoscopy, and have a colorectal cancer diagnosed and removed. As the patient is considered at high risk for developing recurrent colorectal neoplasia in the future, the man would be monitored serially. A metastatic lesion is subsequently detected that is associated with a detection of MMP RNA in the plasma. The man is thus started on a matrix metalloproteinase inhibitor therapy. Serial evaluation of quantitative MMP RNA levels in plasma is undertaken to evaluate response to the therapeutic regimen. MMP RNA levels demonstrate progressive decline into the range for a normal population during the treatment period, indicating a good response to therapy.

This example demonstrates use of the invention for detection and monitoring of neoplasia, and determining drug treatment. Furthermore, the example demonstrates use of the invention in monitoring response to a therapeutic regimen.

1. A method of detecting matrix metalloproteinase (MMP) RNA in blood plasma or serum from a human or animal for detecting, diagnosing, monitoring, treating, evaluating, or determining a prognosis of a neoplastic disease comprising cells that express MMP RNA, the method comprising the steps of:
a) extracting mammalian extracellular RNA from blood plasma or serum;
b) amplifying or signal amplifying a portion of the extracted RNA or cDNA prepared therefrom, wherein said fraction comprises MMP RNA, and wherein amplification is performed qualitatively or quantitatively using primers or probes specific for MMP RNA or cDNA; and
c) detecting the amplified MMP RNA or cDNA product or signal.

2. A method of detecting matrix metalloproteinase (MMP) RNA in a bodily fluid from a human or animal for detecting, diagnosing, monitoring, treating, evaluating, or determining a prognosis of a neoplastic disease comprising cells that express MMP RNA, the method comprising the steps of:
a) extracting mammalian extracellular RNA from a bodily fluid;
b) amplifying or signal amplifying a portion of the extracted RNA or cDNA prepared therefrom, wherein said fraction comprises MMP RNA, and wherein amplification is performed qualitatively or quantitatively using primers or probes specific for MMP RNA or cDNA; and
c) detecting the amplified MMP RNA or cDNA product or signal.

3. The method of claim 2, wherein the bodily fluid is whole blood, blood plasma, serum, urine, effusions, ascites, saliva, cerebrospinal fluid, cervical secretions, endometrial secretions, gastrointestinal secretions, bronchial secretions, or breast fluid, lavages, or aspirations.

4. The method of claim 1, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, signal amplification, amplifiable RNA reporters, Q-beta replication, transcription-based amplification, isothermal nucleic acid sequence based amplification, self-sustained sequence replication assays, boomerang DNA amplification, strand displacement activation, cleavase-based amplification, or cycling probe technology.

5. The method of claim 2, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, signal amplification, amplifiable RNA reporters, Q-beta replication, transcription-based amplification, isothermal nucleic acid sequence based amplification, self-sustained sequence replication assays, boomerang DNA amplification, strand displacement activation, cleavase-based amplification, or cycling probe technology.

6. The method of claim 1, wherein detection of amplified product in step (c) is performed using a detection method that is gel electrophoresis, capillary electrophoresis, ELISA detection using biotinylated or otherwise modified primers, labeled fluorescent or chromogenic probes, Southern blot analysis, Northern blot analysis, electrochemiluminescence, reverse dot blot detection, or high-performance liquid chromatography.

7. The method of claim 2, wherein detection of amplified product in step (c) is performed using a detection method that is gel electrophoresis, capillary electrophoresis, ELISA detection using biotinylated or otherwise modified primers, labeled fluorescent or chromogenic probes, Southern blot analysis, Northern blot analysis, electrochemiluminescence, reverse dot blot detection, or high-performance liquid chromatography.

8. A method of identifying a human or animal having matrix metalloproteinase (MMP) RNA expressing cells or tissue, the method comprising the steps of:
a) extracting mammalian extracellular RNA from a bodily fluid;
b) amplifying or signal amplifying a portion of the extracted RNA or cDNA prepared therefrom, wherein said fraction comprises MMP RNA, and wherein amplification is performed qualitatively or quantitatively using primers or probes specific for MMP RNA or cDNA; and
c) detecting the amplified MMP RNA or cDNA product or signal.

9. The method of claim 8, wherein the MMP expressing cells or tissue comprise a malignant or premalignant cell or tissue.

10. The method of claim 8, further comprising the step of determining invasive or metastatic potential of a malignancy when the amplified MMP RNA or cDNA product or signal is detected.

11. The method of claim 8, wherein the human or animal has a malignancy or premalignancy.

12. A method according to claim 1, further comprising the step of determining invasive or metastatic potential of a malignancy when the amplified MMP RNA or cDNA product or signal is detected.

13. A method according to claim 2, further comprising the step of determining invasive or metastatic potential of a malignancy when the amplified MMP RNA or cDNA product or signal is detected.

14. A method according to claim 1, further comprising the step of selecting a human or animal for a therapy when the amplified MMP RNA or cDNA product or signal is detected.

15. A method according to claim 2, further comprising the step of selecting a human or animal for a therapy when the amplified MMP RNA or cDNA product or signal is detected.

16. A method according to claim 14, wherein the therapy is administration of a matrix metalloproteinase inhibitor.

17. A method according to claim 15, wherein the therapy is administration of a matrix metalloproteinase inhibitor.

18. A method for detecting matrix metalloproteinase (MMP) RNA, or cDNA reverse-transcribed therefrom, comprising the steps of extracting mammalian extracellular RNA comprising MMP RNA from blood plasma or serum, with or without converting said RNA to cDNA, hybridizing said RNA or cDNA to a detectably-labeled probe specific for MMP RNA or cDNA, and detecting hybridization of MMP RNA or cDNA with the detectably-labeled probe.

19. A method for detecting matrix metalloproteinase (MMP) RNA, or cDNA reverse-transcribed therefrom, comprising the steps of extracting mammalian extracellular RNA comprising MMP RNA from a bodily fluid, with or without converting said RNA to cDNA, hybridizing said RNA or cDNA to a detectably-labeled probe specific for MMP RNA or cDNA, and detecting hybridization of MMP RNA or cDNA with the detectably-labeled probe.

20. A method according to claim 1, wherein the method comprises the additional step of quantitatively or qualitatively comparing the product of MMP RNA produced according to step (b) from plasma or serum of a human to the product of MMP RNA produced according to step (b) from the plasma or serum from a plurality of humans with or without known malignancy or premalignancy.

21. A method according to claim 2, wherein the method comprises the additional step of quantitatively or qualitatively comparing the product of MMP RNA produced according to step (b) from the bodily fluid of a human to the product of MMP RNA produced according to step (b) from the bodily fluid from a plurality of humans with or without known malignancy or premalignancy.

22. A method for detecting a plurality of mammalian extracellular RNA species in a bodily fluid from a human or animal, wherein one mammalian RNA species is a matrix metalloproteinase (MMP) RNA species, the method comprising the steps of:
a) extracting mammalian extracellular RNA from the bodily fluid of a human or animal, wherein said extracted RNA comprises a plurality of mammalian RNA species and wherein one RNA species is an MMP RNA;
b) amplifying or signal amplifying concurrently or sequentially at least one of said plurality of RNA or cDNA produced therefrom, wherein one of said RNA is MMP RNA, to thereby produce an amplified product, wherein amplification is performed qualitatively or quantitatively using primers or probes specific for each RNA species; and
c) detecting the amplified product produced from each amplified RNA species or cDNA produced therefrom.

23. The method of claim 22, wherein the bodily fluid is blood plasma or serum.

24. The method of claim 22, wherein detection of a plurality of RNA species in the bodily fluid is indicative or predictive of malignancy or premalignancy, wherein one RNA species is MMP RNA.

25. A diagnostic kit according to the method of claim 1, comprising matrix metalloproteinase (MMP) RNA or cDNA specific amplification primers or probes.

26. A method according to claim 25, wherein the kit comprises a cDNA chip.

27. A diagnostic kit according to the method of claim 2, comprising matrix metalloproteinase (MMP) RNA or cDNA specific amplification primers or probes.

28. A method according to claim 27, wherein the kit comprises a cDNA chip.

29. The method of claim 1, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

30. The method of claim 2, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

31. The method of claim 8, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

32. The method of claim 18, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

33. The method of claim 19, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

34. The method of claim 22, wherein MMP RNA is MMP-1 RNA, MMP-2 RNA, MMP-3 RNA, MMP-7 RNA, MMP-9 RNA, MMP-10 RNA, MMP-11 RNA, MMP-12 RNA, or MMP-14 RNA.

35. The method of claim 19, wherein the bodily fluid is whole blood, blood plasma, serum, urine, effusions, ascites, saliva, cerebrospinal fluid, cervical secretions, endometrial secretions, gastrointestinal secretions, bronchial secretions, or breast fluid, lavages, or aspirations.

36. The method of claim 22, wherein the bodily fluid is whole blood, blood plasma, serum, urine, effusions, ascites, saliva, cerebrospinal fluid, cervical secretions, endometrial secretions, gastrointestinal secretions, bronchial secretions, or breast fluid, lavages, or aspirations.
